(19) United States
(12) Patent Application Publication
(10) Pub. No.: US 2004/0219390 A1
(43) Pub. Date: Nov. 4, 2004

(54) Benzoxazinone and quinazolinone derivatives

(75) Inventors: Thomas Potrawa, Seelze (DE); John Magno, Middletown, NJ (US); Ilyas Khayrullin, Flanders, NJ (US); Susanne Hoyer, Gehrden (DE)

Correspondence Address: HONEYWELL INTERNATIONAL INC., 101 COLUMBIA ROAD, P O BOX 2245, MORRISTOWN, NJ, 07962-2245, US

(73) Assignee: Honeywell International, Inc., Morristown, NJ (US)

(21) Appl. No.: 10/763,792

(22) Filed: Jan. 23, 2004

Related U.S. Application Data
(60) Provisional application No. 60/442,037, filed on Jan. 23, 2003.
(60) Provisional application No. 60/442,230, filed on Jan. 24, 2003.

(51) Int. Cl. H05B 33/14; C09K 11/06; C07D 239/86; C07D 265/22; C07D 413/02

(52) U.S. Cl. 428/690; 428/917; 313/504; 313/506; 252/301.16; 257/102; 544/73; 544/253; 544/296

(57) ABSTRACT

Benzoxazinone and quinazolinone compounds useful in organic light emitting devices (OLEDS) are described.

This application claims priority under 35 U.S.C. § 119(e) from U.S. Provisional application No. 60/442,037, filed on Jan. 23, 2003, titled Method and System For Fabricating An OLED; and U.S. Provisional application No. 60/442,230, filed on Jan. 24, 2003 titled Benzoxazinone and Quinazolinone Derivatives, and under 35 U.S.C. § 120 from U.S. application Ser. No. 10/738,558 filed on Dec. 17, 2003, the entire disclosures of each of which are hereby incorporated by reference herein.

### 1. FIELD OF THE INVENTION

The present invention relates generally to substituted benzoxazinone and quinazolinone derivatives, and, more particularly, relates to the use of these compounds in an organic light emitting device.

### 2. BACKGROUND OF THE INVENTION

Tang and Van Slyke first reported on the electroluminescent properties of multi-layer devices using an organic material in 1987. C. W. Tang and S. A. Van Slyke, “Organic Electroluminescent Diodes,” Appl. Phys. Lett. 51, pp. 913-915 (1987). Various organic light emitting devices have been developed since that time. A great amount of interest has been generated because of their potential for use in a wide variety of applications.

Organic electroluminescent devices are a class of optoelectronic devices in which light emission is produced in response to an electrical current through the device. The terms “organic light emitting diode”, “organic light emitting display” or “organic light emitting device” (OLED) are commonly used to describe an organic electroluminescent device where the current-voltage behavior is non-linear. As used herein, the term “OLED” or “OLED device” refers to this class of devices.

Unlike liquid crystal displays (LCDs) which typically require backlighting and modulate transmitted or reflected light, OLED displays are emissive devices, i.e., intense light is emitted. As a result, OLED displays are brighter, thinner, and lighter, require less space and power, offer higher contrast, and are cheaper to manufacture than LCDs. A large area display device with low-voltage driving is possible with an OLED.

In a single layer arrangement, an OLED includes an organic emissive layer, typically a spin-coated conjugated polymer, disposed between two electrodes. In a bi-layer arrangement (also referred to as single heterostructure), an OLED includes two organic layers composed of small molecules that are sequentially deposited in forming a stack structure. The two organic layers are disposed between an anode and a cathode. In the bi-layer arrangement, one of the layers is multi-functional and operates as both an emissive layer and as an electron transporting layer, or as an emissive layer and as a hole transporting layer. The other layer is a hole transporting or electron transporting layer, respectively.

In a multi-layer arrangement (also referred to as double heterostructure), an OLED includes several organic layers disposed between an anode and a cathode in the resulting stack structure. In the multi-layer arrangement, a separate emissive layer is disposed between a separate electron transport layer and hole transport layer. The separate emissive layer is typically an organic light emitting material or a mixture thereof in the form of a thin amorphous or crystalline film disposed between the hole transport layer and the electron transport layer. The emissive layer composed of an organic material can be made to electroluminesce by applying voltage across the device.

By applying voltage with sufficient amplitude and polarity to the OLED, the anode injects positive charge carriers (holes) and the cathode injects negative charge carriers (electrons), which undergo electron-hole pair recombination then radiatively decay, and in so doing, emit a photon. It should be understood by those with ordinary skill in the art that radiative decay and non-radiative decay may result in emission, or non-emission, respectively, of a photon. In a bi-layer device, the holes and electrons recombine at the interface of the emission/hole-transport layer or the emission/electron transport layer, referred to herein as a recombination zone, which can extend beyond the interface to include regions of the adjacent hole transport layer or the electron transport layer. In a multi-layer device, the holes and electrons recombine at the recombination zone of the emissive layer, which can likewise extend into adjacent and surrounding layers, such as the electron transport layer and the hole transport layer.

As the recombined excited molecules radiatively and nonradiatively decay, energy is released and emitted in the form of a photon thereby generating light, commonly referred to as electroluminescence. Electroluminescence is understood to be produced by the recombination of holes and electrons in the electron transporting layer recombination zone of a bi-layer structure, and in the separate emissive layer recombination zone of a multi-layer device. By selectively choosing the proper materials in fabricating an OLED, a significant quantity of photons are emitted, thereby generating light.

One of the goals in designing the arrangement and composition of the organic layers, in addition to the choice of materials for the anode and cathode, is to maximize the recombination process in the area of the emissive layer, thereby maximizing the light output from the OLED. Since the intensity is directly proportional to the current density through the device, the thin layer construction of about 1000 to about 2000 Angstroms of the bi-layer or multi-layer devices allows the device to operate with a low voltage, i.e., about 2-10 V.

Desirable characteristics of an OLED include brightness, an extended operating lifetime, durability, electroluminescence efficiency, power efficiency, and a broad range of vibrant colors defined by the desired application. Attaining desirable characteristics in a multi-layer OLED device composed of different chemical compounds with all interface boundaries and doping concentrations greatly depends on the fabrication methods and processes involved, in addition to large-scale manufacturing concerns. Various desirable emission colors may not be readily obtainable due to the above limitations in fabrication and manufacturing methods.

Compounds which emit red or green light are readily available, but compounds which emit blue light are less common. As compared to the available compounds which emit red and green light in an OLED, the available compounds which emit blue light do not provide for an extended lifetime in a device in which they are incorporated. Thus, performance levels of blue light emitting devices remain below expectations. The compounds also tend to compromise luminance efficiency, as the known blue emitting compounds incorporated into an OLED device tend to fade or change color, which in turn, limits the utility of displays for many applications. Other shortcomings related to blue emitting compounds include poor emission hue, low luminance, and poor operational stability. Some blue light emitting compounds require high operation voltages. An OLED device incorporating blue light emitting compounds typically possesses limited lifetime as a result.

Applications for which OLEDs are useful include displays for high performance devices, including computer displays, monitors, notebooks, and television screens, flat panel displays, and general lighting elements, including, for example, instrumentation panels used in the automotive, aerospace, military, medical and other industrical applications, in addition to use as light sources, such as in bulbs, small displays for cellular phones, microdisplays for wearable computers and electronic game applications, view-finders in videocamcorders, and electronic books and newspapers, and other consumer electronic devices. Other uses include ink jet printing, bar code tags, digital video cameras, digital versatile disk (DVD) players, personal digital assistants (PDAs), stereos, and other personal products. OLED devices advantageously operate over a broad range of temperature conditions and over a wider viewing angle (about 160 degrees) than LCDs in the above-mentioned devices.

There remains a need for improved luminescent compounds and compositions which emit blue light for used in organic electroluminescent (EL) devices, including OLEDS, which were heretofore unavailable for use in an OLED. There remains also a need for compounds and compositions which provide a full range of brilliant colors for use in fabricating an OLED, which compounds were heretofore unavailable for use in an OLED, particularly those which emit blue light.

### SUMMARY OF THE INVENTION

Briefly described, the present invention provides an organic light emitting device includes an emitting layer, which emitting layer includes a compound according to Formula 1:

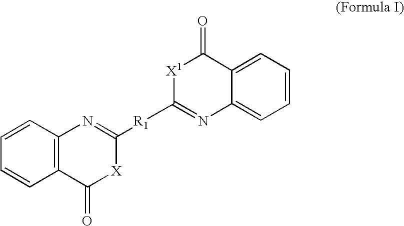

R₁ represents a bond; or from 1 to 3 ring structures, each independently selected from the group consisting of: monocyclic aryls containing 3 to 10 carbon atoms, multicyclic aryls containing 7 to 14 carbon atoms, each monocyclic aryl and multicyclic aryl optionally substituted with one or more hydroxy, halo, acetoxy, or 4H-3,1-benzoxazin-4-one groups; 4 to 10 ring member monocyclic heteroaryls, and 6 to 14 ring member multicyclic heteroaryls, each monocyclic heteroaryl and multicyclic heteroaryl optionally substituted with one or more hydroxy, halo or acetoxy groups.

The monocyclic aryl is preferably a phenyl ring, optionally substituted with halo, including fluorine, chlorine, iodine, and bromine, or optionally substituted with hydroxy, acetoxy, and/or 4H-3,1-benzoxazin-4-one. The multicyclic aryl is preferably naphthyl, phenanthryl, and anthracenyl. The monoheterocyclic aryl is preferably pyridine and pyrimidine.

Preferred compounds according to the invention include: 2,2′-(1,4-phenylene)bis-4H-3,1-benzoxazin-4-one; 2,2′-(1,4-naphthylene)bis-4H-3,1-benzoxazin-4-one; [2,2′]bi-[benz[d][1,3]oxazinyl]-4,4′-dione; 2,2′,2″-(1,3,5-phenylene)tris-4H-3,1-benzoxazin-4-one; 2,2′-(1,5-pyridyl)bis-4H-3,1-benzoxazin-4-one; 2,2′-(1,3-phenylene)bis-4H-3,1-benzoxazin-4-one; 2,2′-(1,4-naplithylene)bis-4H-3,1-benzoxazin-4-one; 2,2′-(1,4-phenylene)-2,3,5,6-tetrafluoro)bis-4H-3,1-benzoxazin-4-one; 3H, 3′H-[2,2′]-1,4-phenylene-bis-quinazolin-4-one; 2,2′-(1,4-pyridyl)bis-4H-3,1-benzoxazin-4-one; 2,2′-(1,4-phenylene-2,5-diacetoxy)bis-4H-3,1-benzoxazin-4-one; 2,2′-(1,4-phenylene-2,5-dihydroxy)bis-4H-3,1-benzoxazin-4-one; 3H, 3′H-[2,2′]-biquinazolinyl-4,4′-dione; and 2,2″-(4,4″-biphenylene)bis-4H-3,1-benzoxazinone.

A method for emitting electromagnetic radiation with at least one wavelength between about 400 and about 550 nm is provided also, and includes the steps of providing an organic light emitting device including an emitting layer interposed between a hole transport layer and an electron transport layer, the hole transport layer is interposed between an anode layer and the emitting layer, and the electron transport layer is interposed between a cathode layer and the emitting layer, wherein at least one of the anode layer and cathode layer is transparent. The emitting layer includes a compound according to Formula 1. An electrical potential is applied to the device between the cathode and anode so that the compound of the emitting layer emits electromagnetic radiation.

The invention provides also an organic light emitting device containing an emitting layer interposed between two electrodes, one of the electrodes being transparent, and the device includes an anode electrode layer, a hole transport layer disposed directly on the anode layer, an emitting layer disposed directly on the hole transport layer, the emitting layer including a compound according to Formula 1, an electron transport layer disposed directly on the emitting layer, and a cathode electrode disposed directly on the electron transport layer.

The compound according to Formula I preferably includes 2,2′-(1,4-phenylene)bis-4H-3,1-benzoxazin-4-one (1,4 PPO); 2,2′-(1,4-naphthylene)bis-4H-3,1-benzoxazin-4-one (1,4 NBBO); [2,2′]bi-[benz[d][1,3]oxazinyl]-4,4′-dione (2,2 BBO); 2,2′,2″-(1,3,5-phenylene)tris-4H-3,1-benzoxazin-4-one (1,3,5 PTBO); 2,2′-(1,5-pyridyl)bis-4H-3,1-benzoxazin-4-one (1,5 PyBBO); 2,2′-(1,3-phenylene)bis-4H-3,1-benzoxazin-4-one (1,3 PBBO); 2,2′-(1,4-naphthylene)bis-4H-3,1-benzoxazin-4-one (2,6 NBBO) 2,2′-(1,4-phenylene)-2,3,5,6-tetrafluoro)bis-4H-3,1-benzoxazin-4-one (1,4 PTFBBO); 3H, 3′H-[2,2′]-1,4-phenylene-bis-quinazolin-4-one (1,4 PBQO); 2,2′-(1,4-pyridyl)bis-4H-3,1-benzoxazin-4-one (1,4 PyBBO); 2,2′-(1,4-phenylene-2,5-diacetoxy)bis-4H-3,1-benzoxazin-4-one (1,4 PDABBO); 2,2′-(1,4-phenylene-2,5-dihydroxy)bis-4H-3,1-benzoxazin-4-one (1,4 PDHBBO); 3H, 3′H-[2,2′]-biquinazolinyl-4,4′-dione (2,2 BQO); and 2,2′-(4,4′-biphenylene)bis-4H-3,1-benzoxazin-4-one (4,4 BPBBO).

The emitting layer can include a compound according to Formula I doped in a host compound, such as 4,4″-N,N′-dicarbazole-biphenyl (CBP), with a dopant-to-host ratio of 0.5-5 weight percent (wt. %) to 99.5-95 wt. %, respectively. This ratio of dopant to host may also be less than 0.5 wt. % or higher than 5 wt. %, depending on the device performance and the targeted specifications related to the particular application. The host compound is not limited to 4,4″-N,N′-dicarbazole-biphenyl, but includes any suitable wide energy band gap organic compound suitable for use as a host compound in an OLED. According to an aspect of the invention, the emitting layer preferably consists of 2,2′-(1,4-phenylene)bis-4H-3,1 -benzoxazin-4-one (1,4 PPO) and 4,4″-N,N′-dicarbazole-biphenyl (CBP) in ratios of 2 wt. % to 98 wt. % and 3.9 wt. % to 96.1 wt. %, respectively According to an aspect of the invention, the emitting layer preferably consists of 2,2′,2″-(1,3,5-phenylene)tris-4H-3,1-benzoxazin-4-one (1,3,5 PTBO) and 4,4″-N,N′-dicarbazole-biphenyl (CBP) in ratio of 1.18 wt. % to 98.82 wt. %, respectively According to an aspect of the invention, the emitting layer preferably consists of [2,2′]bi-[benz[d][1,3]oxa-zinyl]-4,4′-dione (2,2 BBO) and 4,4″-N,N′-dicarbazole-biphenyl (CBP) in ratio of 1.4 wt. % to 98.6 wt. %, respectively. According to an aspect of the invention, the emitting layer preferably consists of 2,2′-(1,3-phenylene)bis-4H-3,1-benzoxazin-4-one (1,3 PBBO) and 4,4″-N,N′-dicarbazole-biphenyl (CBP) in ratio of 1,1 wt. % to 98.9 wt. %, respectively. According to an aspect of the invention, the emitting layer preferably consists of 3H, 3′H-[2,2′]-1,4-phenylene-bis-quinazolin-4-one (1,4 PBQO) and 4,4″-N,N′-dicarbazole-biphenyl (CBP) in ratio of 1,4 wt. % to 98.6 wt. %, respectively. According to an aspect of the invention, the emitting layer preferably consists of 2,2′-(1,4-phenylene)-2,3,5,6-tetrafluoro)bis-4H-3,1-benzoxazin-4-one (1,4 PTFBBO) and 4,4″-N,N′-dicarbazole-biphenyl (CBP) in ratio of 1.96 wt. % to 98.16 wt. %, respectively. According to an aspect of the invention, the emitting layer preferably consists of 2,2′-(1,4-naphthylene)bis-4H-3,1-benzoxazin-4-one (1,4 NBBO) and 4,4″-N,N′-dicarbazole-biphenyl (CBP) in ratio of 1.3 wt. % to 98.7 wt. %, respectively. According to an aspect of the invention, the emitting layer preferably consists of 2,2′-(4,4′-biphenylene)bis-4H-3,1-benzoxazin-4-one (4,4 BPBBO) and 4,4″-N,N′-dicarbazole-biphenyl (CBP) in ratio of 2.3 wt. % to 97.7 wt. %, respectively. According to an aspect of the invention, the emitting layer preferably consists of 2.2′-(1,4-naphthylene)bis-4H-3,1-benzoxazin-4-one (2.6 NBBO) and 4,4″-N,N′-dicarbazole-biphenyl (CBP) in ratio of 1.3 wt. % to 98.7 wt. %, respectively.

The invention provides also a method for manufacturing the device, including providing a substrate layer, disposing a first electrode layer directly on the substrate layer, disposing a hole transport layer directly on the first electrode layer, disposing a emitting layer directly on the hole transport layer, the emitting layer including a compound according to Formula I, disposing an electron transport layer directly on the emitting layer, and, disposing a second electrode layer directly on the electron transport layer, one of the first electrode layer and the second electrode layer being transparent.

### BRIEF DESCRIPTION OF THE DRAWINGS

The detailed description of the invention is more fully understood when read in conjunction with the FIGURES, which include:

FIG. 1a is a cross-sectional view of an OLED according to an aspect of the invention;

FIG. 1b is an exploded view of an OLED according to an aspect of the present invention;

FIG. 1c is a cross-sectional view of an OLED according to an aspect of the present invention;

FIG. 2a illustrates a plot of the current density vs. voltage for an OLED according to an aspect of the invention;

FIG. 2b illustrates the dependence of device luminance upon the current density;

FIG. 2c compares the device electroluminescence spectrum to the photoluminescence spectrum of 1,3PBBO film deposited upon blank glass substrate;

FIG. 3 illustrates the electroluminescence spectra of a series of OLEDs according to an aspect of the invention comprising 2,2′-(1,3-phenylene)bis-4H-3,1-benzoxazin-4-one (1,3 PBBO) as an emitter compound doped into a CBP host compound;

FIG. 4 illustrates the relationship between electroluminescence color coordinates and the dopant concentration for the series of OLEDs according to FIG. 3;

FIG. 5a illustrates a plot of the current density and the luminance vs. voltage for an OLED according to an aspect of the invention comprising 2,2′-(1,4-phenylene)bis-4H-3,1-benzoxazin-4-one (1,4 PPO) as an emitter compound doped into a CBP host;

FIG. 5b illustrates the electroluminescence spectrum of the device;

FIG. 6a illustrates a plot of the current density vs. voltage for an OLED according to an aspect of the invention comprising 2,2′-(1,4-naphthylene)bis-4H-3,1-benzoxazin-4-one (2,6 NBBO) as an emitter compound doped into a CBP host;

FIG. 6b illustrates the dependence of the device luminance upon the current density;

FIG. 6c compares the electroluminescence spectrum of the device to the photoluminescence spectrum of 2,6 NBBO film deposited upon blank glass substrate;

FIG. 7a illustrates a plot of the current density vs. voltage for an OLED according to an aspect of the invention comprising 2,2′-(4,4′-biphenylene)bis-4H-3,1-benzoxazin-4-one (4,4 BPBBO) as an emitter compound doped into a CBP host this device;

FIG. 7b illustrates the dependence of the device luminance upon the current density; and

FIG. 7c compares the electroluminescence spectrum of the device to the photoluminescence spectra of 4,4 BPBBO film deposited upon blank glass substrate and a 4,4 BPBBO bulk sample.

### DETAILED DESCRIPTION OF THE INVENTION

The benzoxazinone and quinazolinone compounds of the invention may be prepared according to processes disclosed in the following patents and publications: U.S. Pat. Nos. 5,560,852, 3,989,698, and 3,408,326, and Synthesis of 2-Substituted-4H-3,1-benzoxazinones, Bain, D. I., et al., J.Chem.Soc. C (13), pp. 1593-1597 (1968), the entire disclosures of each of which are hereby incorporated by reference. The compounds thus prepared provide thermal and chemical stability, and are easy to manufacture.

OLEDS are fabricated, by way of example, using the method and system according to U.S. Provisional Patent Application Ser. No. 60/434,012, filed on Dec. 17, 2002, U.S. Provisional application No. 60/442,037, filed on Jan. 23, 2003, and U.S. Provisional application No. 60/442,230, filed on Jan. 24, 2003, the entire disclosures of each of which are hereby incorporated by reference herein.

Preferred compounds according to the present invention include the following: 2,2′-(1,4-phenylene)bis-4H-3,1-benzoxazin-4-one (1,4 PPO); 2,2′-(1,4-naphthylene)bis-4H-3,1-benzoxazin-4-one (1,4 NBBO); [2,2′]bi-[benz[d][1,3]oxazinyl]-4,4′-dione (2,2 BBO); 2,2′,2″-(1,3,5-phenylene)tris-4H-3,1-benzoxazin-4-one (1,3,5 PTBO); 2,2′-(1,5-pyridyl)bis-4H-3,1-benzoxazin-4-one (1,5 PyBBO); 2,2′-(1,3-phenylene)bis-4H-3,1-benzoxazin-4-one (1,3 PBBO); 2,2′-(1,4-naphthylene)bis-4H-3,1-benzoxazin-4-one (2,6 NBBO); 2,2′-(1,4-phenylene)-2,3,5,6-tetrafluoro)bis-4H-3,1-benzoxazin-4-one (1,4 PTFBBO); 3H, 3′H-[2,2′]-1,4-phenylene-bis-quinazolin-4-one (1,4 PBQO); 2,2′-(1,4-pyridyl)bis-4H-3,1-benzoxazin-4-one (1,4 PyBBO); 2,2′-(1,4-phenylene-2,5-diacetoxy)bis-4H-3,1-benzoxazin-4-one (1,4 PDABBO); 2,2′-(1,4-phenylene-2,5-dihydroxy)bis-4H-3,1-benzoxazin-4-one (1,4 PDHBBO) 3H, 3′H-[2,2′]-biquinazolinyl-4,4′-dione (2,2 BQO); and 2,2″-(4,4″-biphenylene)bis-4H-3,1-benzoxazinone (4,4 BPBBO).

The compounds may be deposited using thermal vacuum deposition techniques, which provides the advantages of making a smooth surface, minimizing the impurity of a thin film occurring in the spin-coating method, and also aids in controlling the film thickness. The compounds may also be deposited using pulsed laser deposition, according to the method disclosed in U.S. provisional patent application Ser. No. 60/434,012, filed on Dec. 17, 2002 and U.S. Provisional application No. 60/442,037, filed on Jan. 23, 2003, the entire disclosures of each of which are hereby incorporated herein by reference.

The compounds according to the invention may be suitably used in substantially any type of device that includes an OLED. For example, OLEDs that include a compound or composition according to an aspect of the invention may be incorporated into a display, vehicle, computer, television, printer, theater or other large display screen or sign.

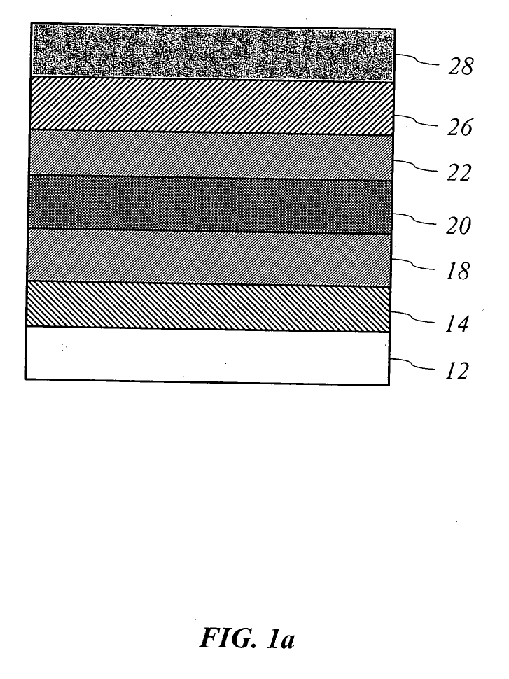

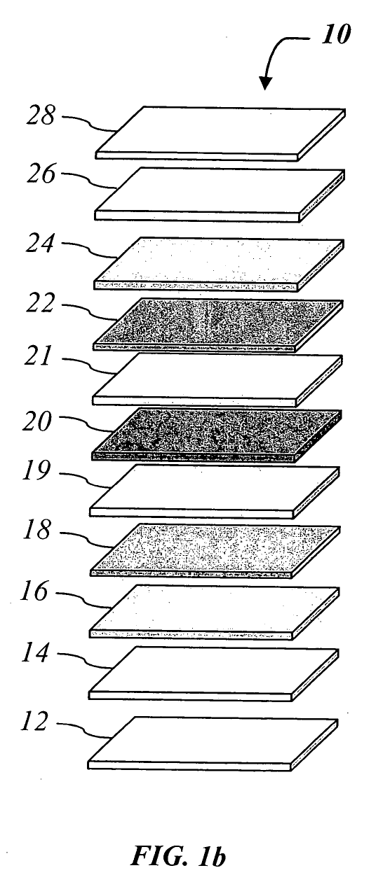

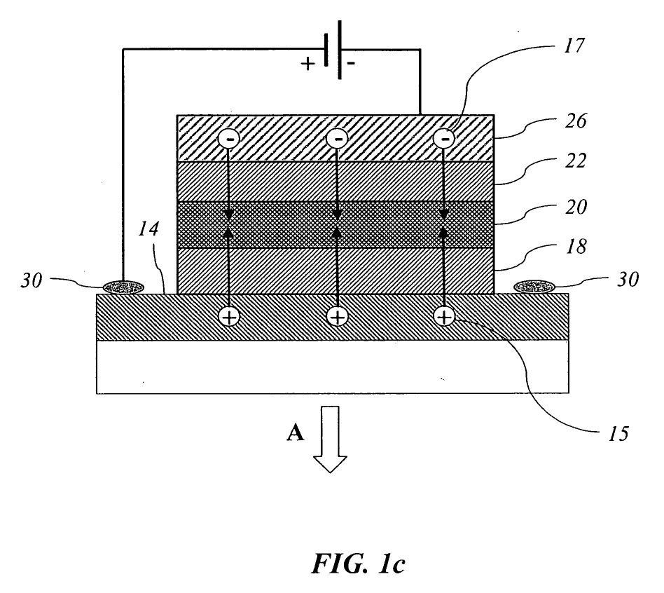

An OLED device 10 includes organic layers 16, 18, 19, 20, 21, 22, and 24 that are sequentially deposited thereby forming a stack structure, as illustrated in FIGS. 1a and 1b. The stack may take the form of a bi-layer (single heterostructure) or a multi-layer (double heterostructure) arrangement. The functions of the organic layers are distinct and are addressed independently herein, as each may be optimized according to various desired properties, for example, obtaining a desired color or high luminance efficiency. For visual display applications, organic luminescent material should provide a satisfactory color in the visible spectrum, normally with emission maxima at about 460, 550, and 630 nm for blue, green, and red, respectively. Light generating applications include but are not limited to general light sources, bulbs, screens, design elements, and street lights. Colors may be of any hue with a maximum at any wavelength of the visible spectrum. Light emission at wavelengths ranging from about 400 nm to about 700 nm, and preferably from about 400 nm to about 550 nm, the latter of which can be described as blue and blue-green, are advantageously achieved with the OLEDs according to the invention.

It is to be understood that there may be substantial variations in the type, number, thickness, order, arrangement, and composition of the layers of an OLED device, depending upon the desired application. With regard to total device thickness, the device must be thin enough to work at low voltage. It is also to be understood that the invention is not to be limited to or bound by the theories of operation set forth herein.

It is to be further understood that the materials used in the fabrication of an OLED device are chosen based on their respective ability to transport and inject holes, transport and inject electrons, to block the flow of electrons or holes, and to electroluminesce. The invention is not limited to any particular material, provided that the function of injecting holes, transporting holes, and injecting and transporting electrons, blocking the flow of electrons or holes, is met for use in a layer of an OLED with the material selected. Efficiency of carrier-injection can be improved by choosing organic hole injection layers with a low HOMO (highest occupied molecular orbital) or a high LUMO (lowest unoccupied molecular orbital). It is to be understood that the materials used according to an aspect of the invention in the emission layer of the OLED device include all materials that luminesce by way of singlet excitation or triplet excitation, or by both excitations, i.e., fluorescence and/or phosphorescence.

The color of light emitted by the molecules depends upon the energy difference between the ground and excited states. According to an aspect of the invention, the blue and blue-green light emitting compounds and compositions emit a blue and blue-green light. Depending upon the amount of blue and blue-green light emitting compound used in doping the emitting layer, or the particular host selected for use in the emitting layer, a device 10 incorporating the compounds according to the invention may emit light in another wavelength.

Referring to FIGS. 1a, 1b and 1c, an OLED device 10 according to an aspect of the invention preferably includes an anode layer 14 disposed onto the surface of a transparent substrate 12, a hole transport layer (HTL) 18, an emitting layer (EML) 20, an electron transport layer (ETL) 22, and a cathode layer 26. A protective layer 28 composed of glass or plastic is typically disposed adjacent the cathode layer 26, as illustrated in FIGS. 1a and 1b, to protect against oxidation and moisture, and is held in place with a suitable adhesive, for example, a UV curable adhesive. The protective layer 28 may be composed of any suitable material, conductive or non-conductive. Other alternative preferred embodiments include additional layers, i.e., a hole injection layer (HIL) 16, an electron blocking layer (EBL) 19, a hole blocking layer (HBL) 21, and an electron injection layer (EIL) 24, as illustrated in FIG. 1b. As discussed above, it is to be understood that the number, arrangement, and composition of the individual layers of the device 10 may be selectively varied.

Referring to FIG. 1c, an electrical potential difference is applied between the cathode 26 and the contacts 30, for example, pogo pins plated with silver, disposed on the anode 14, whereby holes (positive charge carriers) 15 are injected by the anode 14 and migrate across the hole injection layer 16 (when present) and the hole transport layer 18 to the region of the emitting layer 20. Likewise, the electrons (negative charge carriers) 17 injected by the cathode 26 migrate across the electron injection layer 24 (when present) and the electron transport layer 22 to the region of the emitting layer 20. Injected electrons and holes are mobile, and migrate under the influence of an applied field toward the oppositely charged electrode. In this embodiment, the holes recombine with the electrons in the region of the adjacent emitting layer 20. Light is emitted in the direction of the arrow A. The potential applied to the anode 14 is higher than the potential applied to the cathode 26, and a low voltage of a few Volts, such as about 2 to 10, or up to about 20 Volts, is sufficient to drive enough current to the OLED to achieve a very bright and intense light emission.

Referring to FIG. 1a, an OLED device 10 according to an aspect of the invention includes a substrate 12 composed of, for example, glass. An electrode (anode) 14 is disposed adjacent the substrate 12. Substrate 12 and the anode 14 should be transparent, i.e., the material at a selected thickness is capable of transmitting light at wavelengths emitted by the OLED device 10, and more preferably transmits substantially all of the light emitted. As used herein, a layer of material or several layers of different materials are “transparent” when the layer(s) allow for at least 50% of the ambient electromagnetic radiation in relevant wavelengths to be transmitted therethrough. Substrate 12 may also be composed of quartz, sapphire, or other suitable transparent film material, for example, rigid plastic.

Materials for the anode 14 are preferably those having a high work function. The anode 14 is typically composed of a transparent conductive material, for example, indium tin oxide (ITO), which is formed on a substrate by electron beam deposition, pulsed laser deposition, radio-frequency sputtering, or other known techniques. ITO is typically deposited in a high-temperature sputtering process, and is available from Thin Film Devices in New York or Applied Films in Colorado, or may also be formed in a vacuum deposition chamber according to an aspect of the invention. When depositing on a substrate in a vacuum chamber according to an aspect of the invention, RF magnetron sputtering, along with a shadow mask, is a preferred method in forming an anode pattern on the ITO. The anode should be thin enough to minimize the absorption of light, and thick enough to have low resistivity. The thickness of the deposited anode 14 is from about 200 Angstroms (A) to 1 micron, and preferably is about 1500 Å. Thicknesses outside the above range may also be used. The ITO is subsequently patterned using any suitable technique, for example, etching in the presence of a photoresist layer to remove the resist, and other various known methods to provide conductive areas and non-conductive areas which can be used in electronic circuitry. The ITO layer is then finally cleaned with O₂ plasma, radiofrequency ionic etching, or any other known technique. Although the anode 14 is described as transparent, it should be understood that at least one of the electrodes (cathode or anode) should be optically transparent to allow for transmission of light visible to an observer.

The anode 14, which injects holes 15 into the hole transporting layer 18, should have a high work function and an energy close to that of the HOMO levels of the hole transporting molecules. A preferred material for the anode 14 is ITO as a source for emitting holes 15 into the highest occupied molecular orbital (HOMO) levels of the hole-transporting molecules. ITO is preferred due to its high work function, i.e., a large amount of energy (4.7-5.0 eV) is required to remove an electron. Other suitable materials for emitting holes, however, may be employed in the present invention, for example, gallium indium tin oxide (GITO), and zinc indium tin oxide (ZITO).

As illustrated in FIGS. 1a and 1b, the device 10 also includes a hole transport layer (HTL) 18, and may optionally include a hole injection layer (HIL) 16. The HTL 18 is preferably disposed at a thickness ranging from about 300 to 800 Angstroms, at a rate of about 1 Å/s up to 10 Å/s, with an average rate of about 2 Å/s onto the upper surface of the anode 14. Most HTL materials are based on aromatic amines, known for their high hole mobility as compared to other organic molecules. Suitable HTL materials have a low ionization potential with a small electron affinity associated with a large energy gap. Compounds preferred for use as a hole transport layer 16 include metal phthalocyanines, such as copper phthalocyanine (CuPc), carbonyl compounds such as 1,1,4,4-tetraphenyl-1,3-butadiene (TPB), arylamines, such as N,N′-diphenyl-N,N′-bis(3-methylphenyl)-1,1′biphenyl-4,4′diamine (TPD), naphthyl-substituted benzidine derivatives, for example, 4,4′-bis[N-(1-naphthyl)-N-phenyl-amino]biphenyl (α-NPD), and N,N′-di-1-naphthyl-N,N′-diphenyl-1,1′-biphenyl-1,1′-biphenyl-4,4′-diamine (α-NPB), which compounds are available from H.W. Sands Corp., of Jupiter, Fla.

In a bi-layer arrangement of an OLED, the HTL injects holes into the combination emission/electron transport layer, where the holes combine with electrons to form excitons. Alternatively, the electrons are injected from the ETL and combine with holes in the combination emission/hole transport layer to form excitons. In either case, the excitons are trapped in the material having the lowest energy gap. Since the ETL typically operates as an emissive layer in the bi-layer OLED, a suitable electron transport material for use in the ETL should have a lower energy gap than the HTL. In the preferred multi-layer arrangement of an OLED, holes 15 are injected from the HTL 18 and electrons 17 are injected from the ETL 22 into the region of the emissive layer 20, where they combine to form excitons. While the HTL injects holes 15 from the anode 14, it also serves to block electrons 17 injected from the cathode 26.

An optional hole injection layer (HIL) 16 illustrated in FIG. 1b is disposed adjacent the anode 14 and the HTL 18, at a thickness ranging from about 50 to 200 Angstroms, at a rate of about 1 Å/s up to 10 Å/s, with an average rate of about 2 Å/s. The HIL 16 may be composed of copper phthalocyanine (CuPc), polyaniline (PANI), or 3,4,9,10-perylentetracarboxylic dianhydride (PTCDA), although other suitable compounds may be used, such as those described with respect to the HTL 18.

The addition of an HIL 16 further improves the brightness and efficiency of the OLED device 10 by lowering the barrier controlling the injection of holes 15. The HIL 16 preferably has a low ionization potential or a high HOMO level between the anode 14 and the HTL 18, which lowers the barrier for injection of holes and lowers also the drive voltage. As with the HTL 18, the HIL 16 assists hole migration toward the emission layer 20.

As illustrated in FIGS. 1a, 1b, and 1c, a light emitting layer (EML) 20 is disposed adjacent ETL 22 and HTL 18. The EML 20 is disposed at a thickness ranging from about 200-500 Å at a deposition rate of about 1 Å/s, up to 10 Å/s, with an average rate of about 2 Å/s. A preferred thickness for EML 20 is about 400 Å. The EML 20 comprises a single dopant compound, or alternatively, may be a composition formed via co-deposition of host and dopant compounds.

According to an aspect of the present invention, EML 20 comprises a single dopant material as herein described, or comprises a host compound doped with the benzoxazinone and quinazolinone compounds herein described. A preferred host material, commonly used as an EML 20, is wide energy bandgap compound carbazole biphenyl (CBP). This compound produces blue electroluminescence. Other preferred host materials for use in the EML 20 include, but are not limited to 2,9-dimethyl-4,7-diphenyl-1,10-phenanthroline, also referred to as bathocuproine (BCP), copper phthalocyanine (CuPc), α-NPB, and 3-(4-biphenylyl)-4-phenyl-5-(4-t-butylphenyl)-1,2,4-triazole (TAZ).

Materials present as host and dopant, particularly in the EML 20, are selected to have a high level of energy transfer between the host and dopant molecules. The term “host” is used to refer to the compound in the emissive layer 20, which receives energy from holes and electrons and changes from its ground state to an excited state. Through an emission/absorption energy transfer process, the excitation energy of the host is then transferred to the dopant emitter compound, which is typically present in a lower concentration than the host compound. Upon receiving the energy from the host, the dopant subsequently changes from its ground state to the excited state. The dopant emitter compound may then relax to the ground state having a slightly lower energy level, which preferentially radiates all of the energy as a luminescent emission in a desired spectral region. The host compound can have a strong emission in a region of the spectrum where the dopant strongly absorbs light, but the host preferably does not have an emission band in a region where the dopant also emits strongly.

The use of host/dopant combinations extends the range of colors emitted by the OLED device 10. According to an aspect of the invention, the range of colors includes blue and blue-green light. Incorporating various dopants into the host compound improves the performance of the device 10, including efficiency and luminance of the host EML 20. The concentration of the dopant depends upon the desired application, and is not restricted to any particular range. An effective amount of dopant present is an amount sufficient to shift the emission wavelength of the host. It is preferred, however, that the concentration of the dopant range from about 0.01 to 10.0 mol %. A preferred amount is 0.1 to about 1.0 mol %, depending upon the host emitting material used. If the dopant concentration is too low, the emission will include a component of light from the host compound. If the dopant concentration is too high, emission efficiencies can be adversely affected by self-quenching, a non-emissive mechanism. Although dopants are described herein for use in the EML 20, they may be used also in the emissive/electron transport layer of a bi-layer device. A host/dopant combination may be present also in other layers of the OLED device 10. Also, the dopants described herein may be used solely as a separate layer of the OLED device 10.

The invention is not limited to any particular ratio of dopant to host. It should be understood that the ratio of dopant to host varies depending upon the particular application. To achieve a 4 wt. % doping level, for example, the deposition rate for the host is maintained at about 9.6 Å/s, and the rate for the dopant is maintained at about 0.4 Å/s. This particular method of deposition achieves a weight ratio of 4:96 for the dopant and host, respectively. The description of deposition rate and thickness herein is for illustration purposes, and should not be construed as limiting as to the particular ratio, speed of deposition, or compounds described. Advantageously, the dopant materials may be deposited or co-deposited with a host compound using pulsed laser deposition, or by conventional thermal vacuum evaporation, according to an aspect of the present invention.

Although a dopant and or host may emit in a particular wavelength, the color emitted by the OLED device 10 can be shifted using a particular dopant or host. For example, the host compound may be CBP, which emits a blue light. If the host layer of CBP is doped with a suitable amount of tris-(4,4,4-trifluor-2-thenoyl-(1,3,-butandionato-O-O′)Europium-di-(triphenylphosphinoxide), the OLED device will emit a red light. Thus, by selectively doping the host compound with a dopant or a plurality of dopants capable of shifting the wavelength of the host, the wavelength of the emissive layer is shifted. It is to be understood that dopants capable of shifting the wavelength of an emissive layer should be present in an amount effective to shift the wavelength to the desired color. Since the color of the light emitted by the molecules depends upon the energy difference between the ground and excited states, the color of the emitted light and the electrical characteristics of the OLED depend upon the specific organic material(s) used. In addition to shifting the emission color of the EML 20, other layers of the OLED device 10 may be doped to achieve changes in emission color or to improve device performance, including efficiency and stability, for example, improving conductivity.

The addition of an electron transport layer (ETL) 22 which is disposed adjacent the cathode 26 and the EML 20 lowers the current density level and also the drive voltage used to operate the device. Compounds described above for use as the EML 20 may also be used in forming an ETL 22. For example, metal chelates of 8-hydroxyquinone, including Alq₃, are preferred electron transport materials. The ETL 22 is deposited with a thickness ranging from about 300 to 500 Angstroms, at a deposition rate of about deposition rate of about 1 Å/s, up to 10 Å/s, with an average rate of about 2 Å/s. A preferred thickness for an EML 20 is about 300 Å to about 500 Å. Thicknesses outside the stated range may also be suitable, depending upon the application.

The device also may optionally include an electron injection layer (EIL) 24 disposed adjacent the ETL 22. The EIL 24 functions to improve injection of electrons from the cathode 26 to the ETL 22. The typical EIL 24 is composed of lithium-fluoride or a calcium compound. The EIL 24 may also comprise any known conventional electron transmitting compound, such as those described herein with regard to the EML 20, and also includes, but is not limited to triazole derivatives, oxazole derivatives, oxadiazole derivatives, fluorenone derivatives, anthraquino-dimethane derivatives, anthrone derivatives, diphenylquinone derivatives, thiopyran dioxide derivatives, carbodiimide derivatives, fluorenylidenemethane derivatives, distyrylpyrazine derivatives, heterocyclic tetracarboxylic anhydrides such as naphthaleneperylene, phthalocyanine derivatives, and various metal complexes represented by metal complexes of 8-quinolinol derivatives, metallophthalocyanine, and metal complexes each having benzoxazole or benzothiazole as a ligand, described in U.S. Pat. No. 6,461,747 to Okada, the entire disclosure of which is hereby incorporated by reference. The thickness of the EIL 24 ranges between about 5 to 40 Å, and is disposed at a deposition rate of about 0.1 Å/s, up to 1 Å/s, with an average rate of about 0.5 Å/s. A preferred thickness is about 10 Å. Thicknesses outside this range may also suitably be used.

The device 10 may also optionally include an electron blocking layer (EBL) 17 and hole blocking layer (HBL) 21. Compounds for use as a HBL 21 include CBP and BCP, in addition to 3,4,5-triphenyl-1,2,4-trizole, 3-(biphenyl-4-yl)-4-phenyl-5-tert-butylphenyl-1,2,4-triazole, 3,5,-bis(4-tert-butylphenyl)-4-phenyl-[1,2,4]triazole, and 2,9-dimethyl-4,7-diphenyl-1,10-phenanthroline, all available from H.W. Sands. Compounds for use as a EBL 17 include TPD.

Materials for the cathode are preferably those having a low work function. A metallic cathode 26, composed of, for example, aluminum, or magnesium and silver, is disposed adjacent the electron injection layer 24. A cathode 26 composed of Al or Mg:Ag, with a ratio of 10:90, respectively, is preferred for use in the present invention. Since the cathode 26 is needed to inject electrons into the LUMO level of the electron-transporting layer(s), material(s) with low work function, for example, from about 3.5 eV to about 4.0 eV, and preferably from about 3.5 eV to about 3.7 eV are used to enhance quantum efficiency. The thickness of the cathode 26 ranges, for example, from about 1000 Angstroms to about 2000 Angstroms. A preferred thickness is about 1500 Angstroms. Thicknesses outside this range may also suitably be used. A protecting layer 28 is also preferably disposed onto the surface of the cathode 26 to protect the device 10 from humidity and environmental conditions.

The OLED devices 10 according to the invention advantageously may be fabricated entirely within a vacuum system using pulsed laser deposition, in combination with other deposition techniques known in the art, without removing the OLED device 10 from the vacuum chamber during fabrication.

The compounds according to the invention may be deposited also using conventional thermal vacuum evaporation techniques.

Pulsed lasers are commercially available within the full spectral range from UV to IR. These lasers typically emit light having a wavelength ranging from about 157 m to 1100 nm, an energy density of about 0.05 to 10 J/cm², a pulse width of about 10⁻¹² to 10⁻⁶ per second, and a pulse repetition frequency of about 0 Hz to greater than 20,000 Hz. Examples of suitable lasers include, but are not limited to pulsed gas lasers, such as excimer lasers, i.e., F2 (157 run), ArF (193 run). KrF (248 nm), XeCl (308 nm), and XeF (351 nm). Other suitable lasers include pulsed solid state lasers, such as YAG (457-1040 nm), and Alexandrite (380 nm to 760 nm). Preferred lasers for use in the present invention are an Alexandrite laser, disclosed in U.S. Pat. Nos. 3,997,853; 4,272,733; 4,734,913; 4,809,283; 4,835,786; 4,858,242; 4,933,946; 4,944,567; 4,949,346; 5,009,658; 5,321,711; and 5,331,652, the disclosures of which are hereby incorporated herein by reference, and an XeCl laser LPX200, manufactured by Lambda Physik of Gottingen, Germany.

According to the method and system described herein, the compounds employed in the invention are capable of being deposited in a vacuum having a background pressure less than one atmosphere, preferably about 10⁻5 to about 10⁻⁹ torr. The method and system of the invention for fabricating an OLED have the advantage that selected aryl benzoxazinone and quinazolinone compounds and compositions may be laser deposited, which, in some instances, cannot be deposited using other techniques without deleteriously altering the structure of the compound. Altering the structure of the compounds by using techniques other than laser deposition can be deleterious to the completed OLED. By employing laser deposition in fabricating OLEDs, less material is consumed than in the aforementioned processes, although the compounds according to the invention may be deposited also using conventional thermal vacuum evaporation techniques.

Preferred stack structures for the OLED devices 10 according to the invention include, but are not limited to the following:

ITO/TPD(400 Å)/CBP+3.9%1,4PPO(400 Å)/BCP(80 Å)/Alq₃(350 Å)/LiF(10 Å)/Al(1915 Å); ITO/TPD(400 Å)/CBP+1.18% 1,3,5PTBO (400 Å)/BCP(100 Å)/Alq₃(350 Å)/LiF(10 Å)/Al(110 Å)/Ag(400 Å); ITO/TPD(400 Å)/CBP+1.4% 2,2 BBO(400 Å)/BCP(100 Å)/Alq₃ (350 Å)/LiF(10 Å)/Al(560 Å)/Ag(500 Å); ITO/TPD(400 Å)/CBP+1.1% 1,3 PBBO(310 Å)/BCP(100 Å)/Alq₃(300 Å)/LiF(10 Å)/Al(680 Å)/Ag(1000 Å); ITO/TPD(400 Å)/CBP+1.4% 1,4PBQO(410 Å)/BCP(100 Å)/Alq₃(350 Å)/LiF(10 Å)/Al(160 Å)/Ag(500 Å); ITO/TPD(400 Å)/CBP+1.96% 1,4PTFBBO (410 Å)/BCP(100 Å)/Alq₃(350 Å)/LiF(10 Å)/Al(200 Å)/Ag(500 Å); ITO/TPD(400 Å)/CBP+1.3%1,4NBBO (400 Å)/BCP(100 Å)/Alq₃(350 Å)/LiF(10 Å)/Al(100 Å)/Ag(500 Å); ITO/TPD(415 Å)/CBP+2.3%4,4BPBBO (400 Å)/BCP(100 Å)/Alq₃(350 Å)/LiF(10 Å)/Al(100 Å)/Ag(500 Å);, and ITO/TPD(400 Å)/CBP+1.3% 2,6NBBO (400 Å)/BCP(100 Å)/Alq₃(350 Å)/LiF(10 Å)/Al(100 Å)/Ag(500 Å).

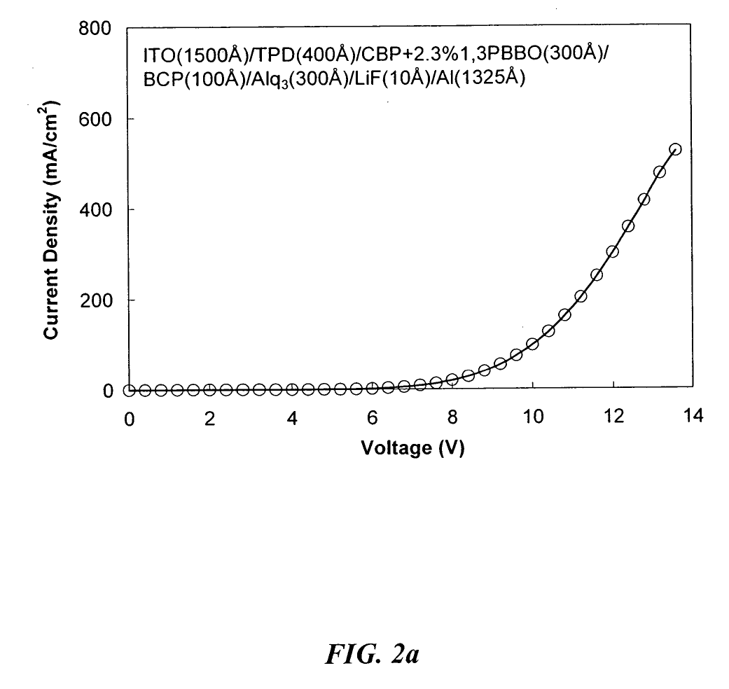

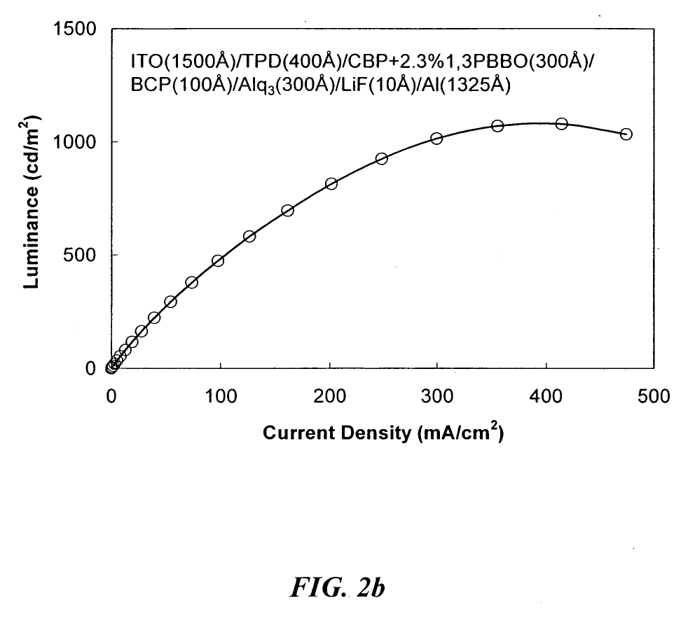

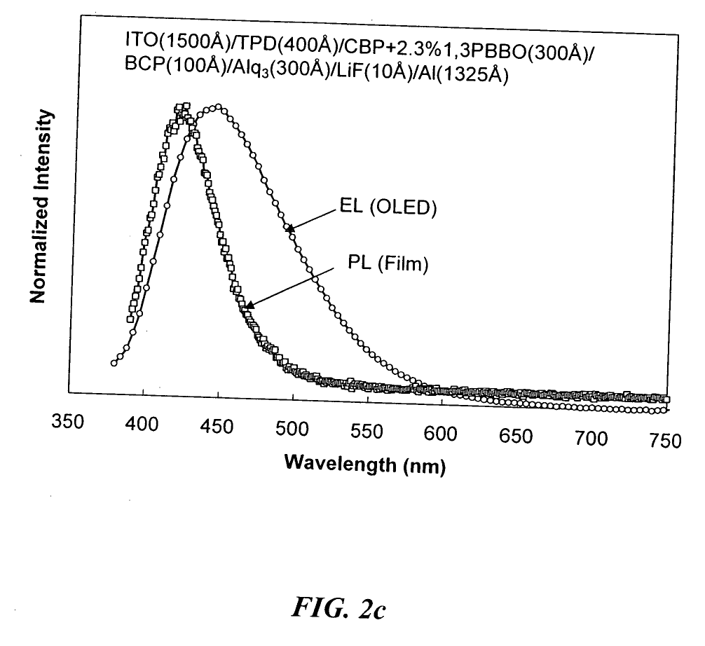

Referring to FIGS. 2a and 2b, the electroluminescence characteristics of an OLED comprising 2,2′-(1,3-phenylene)bis-4H-3,1-benzoxazin-4-one (1,3 PBBO) as an emitter compound doped in a CBP host are illustrated. The stack depicted is ITO(1500 Å)/TPD(400 Å)/CBP+2.3% 1,3 PBBO(300 Å)/BCP(100 Å)/Alq₃(300 Å)/LiF(10 Å)/Al(1325 Å). All layers of the OLED are deposited using conventional thermal vacuum evaporation techniques according to an aspect of the invention. FIG. 2a is a plot of the current density vs. voltage for this device. FIG. 2b illustrates the dependence of the device luminance upon the current density. FIG. 2c compares the electroluminescence spectrum of the device to the photoluminescence spectrum of a 1,3PBBO film deposited upon a blank glass substrate. The “red” wavelength shift and broadening of electroluminescence spectrum can be attributed to the microcavity effect in OLED optical stack.

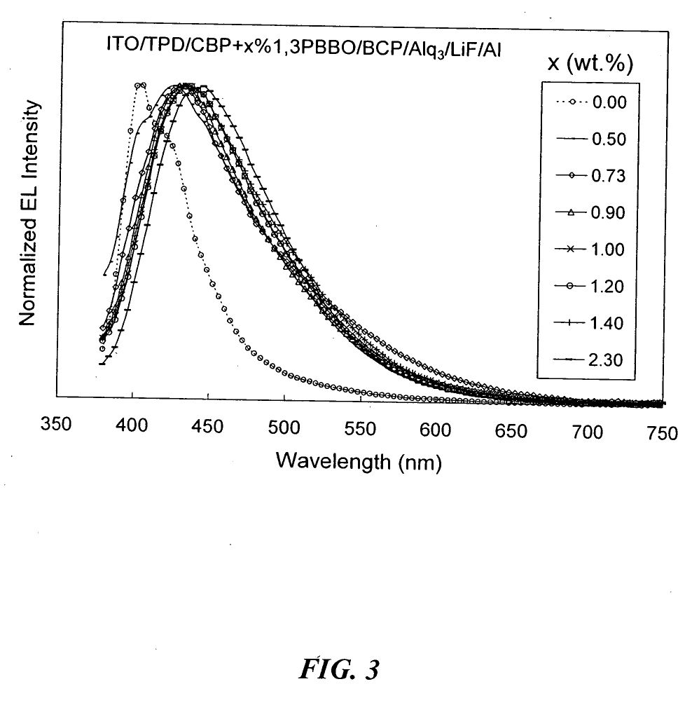

Referring to FIG. 3, the electroluminescence spectra of series of OLEDs with an emitter layer comprising 2,2′-(1,3-phenylene)bis-4H-3,1-benzoxazin-4-one (1,3 PBBO) as the emitter compound doped into a CBP host are illustrated. The doping concentration gradually increased from 0 wt. % to 2.3 wt. % as shown. The stacks depicted are ITO(1500 Å)/TPD(400 Å)/CBP+x % 1,3PBBO(300-400 Å)/BCP(100 Å)/Alq₃(300-350 Å)/LiF(10 Å)/Al(1200-2000 Å), where x is the emitter dopant concentration. All layers are deposited using conventional thermal vacuum evaporation techniques according to an aspect of the invention. While an OLED with an emitter layer of CBP produces light with a peak at 400 nm (characteristic of CBP compound), the devices comprising 1,3PBBO show spectra with a peak gradually shifting from 424 nm (x=0.5 wt. %) to 444 nm (x=2.3 wt. %). The shoulder at 400 nm of the spectrum related to the device with x=0.5 wt. % concentration is referred to as the emission band of the CBP host due to impartial host-to-dopant energy transfer.

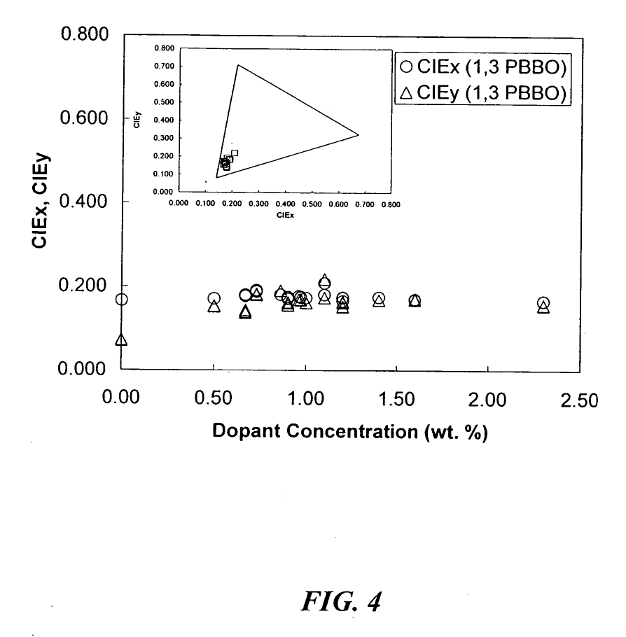

Referring to FIG. 4, the CIEₓ and CIE_{y} color coordinates of series of OLEDs with an emitter layer comprising 2,2′-(1,3-phenylene)bis-4H-3,1-benzoxazin-4-one (1,3 PBBO) as an emitter compound doped into a CBP host are illustrated. The doping concentration gradually increased from 0 wt. % to 2.3 wt. % as shown. The stacks depicted have the same composition as in FIG. 3, where x is the emitter dopant concentration. The inset in FIG. 4 illustrates the same CIEₓ and CIE_{y} data placed into the “NTSC Triangle”, where the lower left corner of the triangle represents a blue color. By changing doping concentrations even in a narrow range such as 0.5-2.3 wt. %, one may substantially vary an OLED's hue.

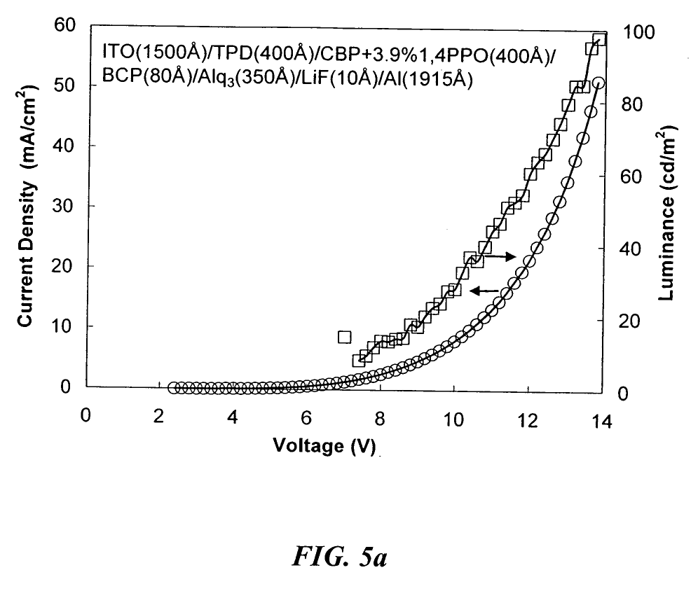

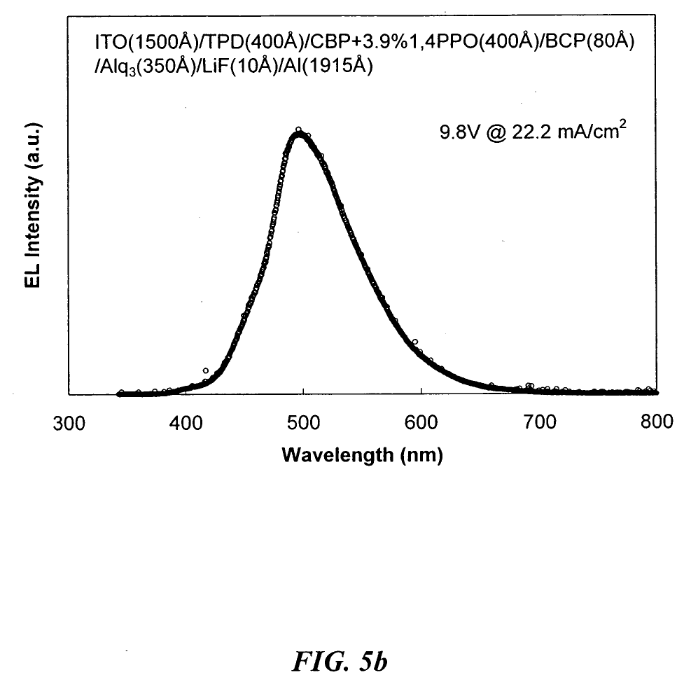

Referring to FIGS. 5a and 5b, the electroluminescence characteristics of an OLED comprising 2,2′-(1,4-phenylene)bis-4H-3,1-benzoxazin-4-one (1,4 PPO) as an emitter compound doped into a CBP host, are illustrated, where the stack is ITO(1500 Å)/TPD(400 Å)/CBP+3.9% 1,4PPO(400 Å)/BCP(80 Å)/Alq₃(350 Å)/LiF(10 Å)/Al(1915 Å). All layers are deposited using conventional thermal vacuum evaporation techniques according to an aspect of the invention. FIG. 5a is a plot of both the current density and luminance vs. voltage for the device, and FIG. 5b illustrates the electroluminescence spectrum of the device.

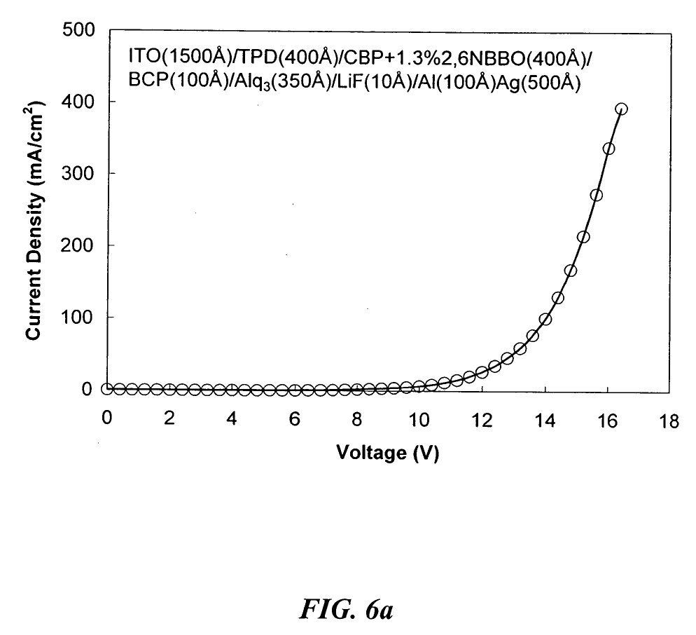

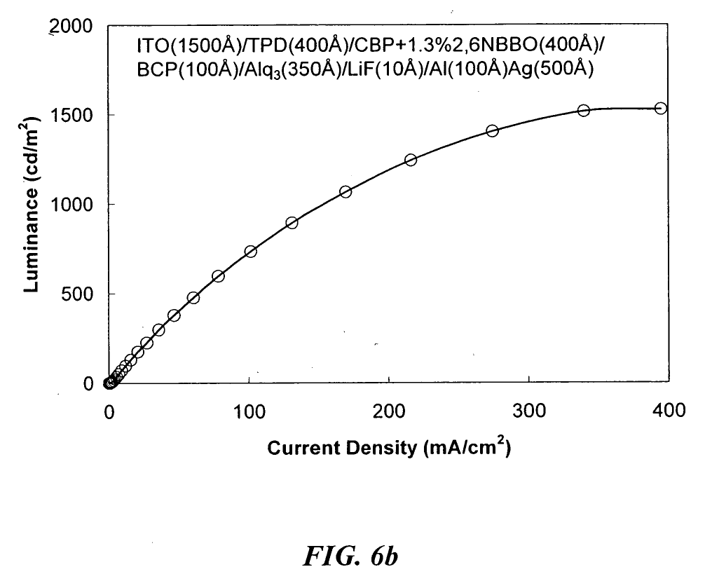

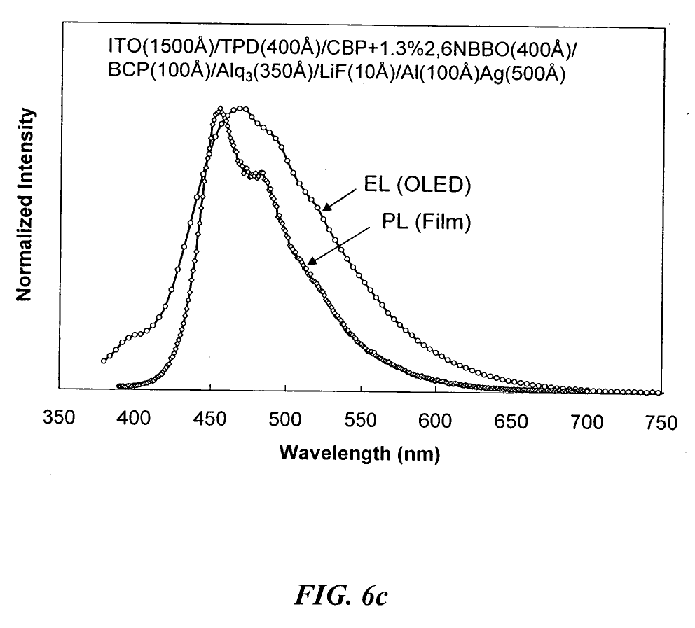

Referring to FIGS. 6a, 6b, and 6c, the electroluminescence characteristics of an OLED comprising 2.2′-(1,4-naphthylene)bis-4H-3,1-benzoxazin-4-one (2,6 NBBO) as an emitter compound doped into a CBP host are illustrated. The stack depicted is ITO(1500 Å)/TPD(400 Å)/CBP+1.3%2,6NBBO(400 Å)/BCP(100 Å)/Alq₃(350 Å)/LiF(10 Å)/Al(100 Å)/Ag(500 Å). All layers are deposited using conventional thermal vacuum evaporation techniques according to an aspect of the invention. FIG. 6a shows a plot of the current density vs. voltage for the device. FIG. 6b illustrates the dependence of the luminance of the device upon the current density. FIG. 6c compares the electroluminescence spectrum of the device to the photoluminescence spectrum of 2,6 NBBO film deposited upon a blank glass substrate. The “red” wavelength shift and broadening of electroluminescence spectrum may be attributed to the microcavity effect in an OLED optical stack.

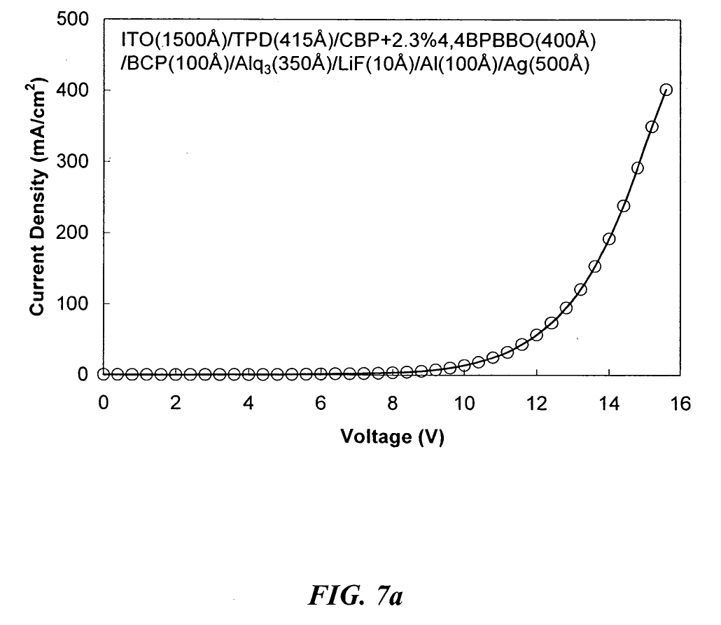

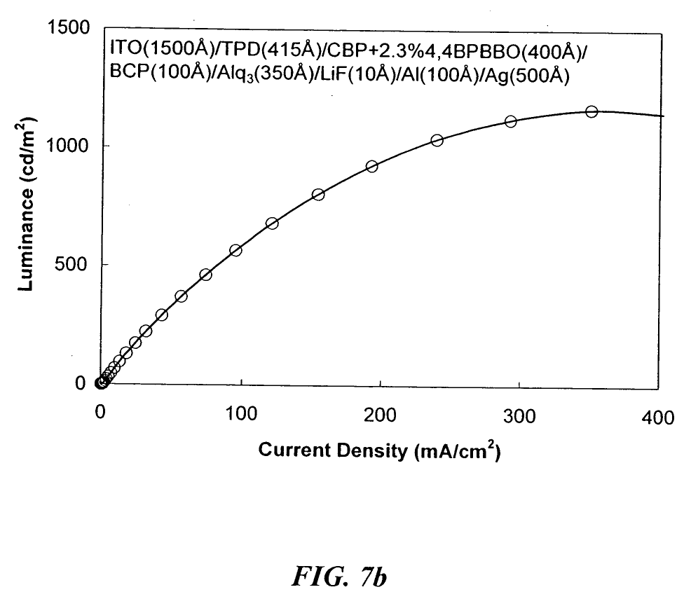

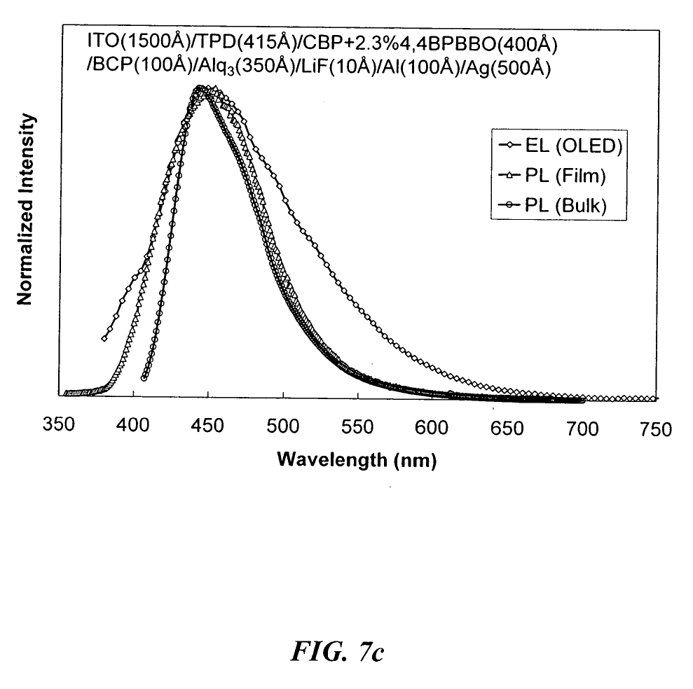

Referring to FIGS. 7a, 7b, and 7c, the electroluminescence characteristics of an OLED comprising 2,2′-(4,4′-biphenylene)bis-4H-3,1-benzoxazin-4-one (4,4 BPBBO) as an emitter compound doped into a CBP host are presented. The stack depicted is ITO(1500 Å)/TPD(415 Å)/CBP+2.3%4,4BPBBO(400 Å)/BCP(100 Å)/Alq₃(350 Å)/LiF(10 Å)/Al(100 Å)/Ag(500 Å). All layers are deposited using conventional thermal vacuum evaporation techniques according to an aspect of the invention. FIG. 7a shows a plot of the current density vs. voltage for the device. FIG. 7b illustrates the dependence of the device luminance upon the current density. FIG. 7c compares the electroluminescence spectrum of the device to the photoluminescence spectra of a 4,4 BPBBO film deposited upon a blank glass substrate and 4,4 BPBBO bulk sample. An apparent broadening of the electroluminescence spectrum can be attributed to the microcavity effect in an OLED optical stack.

TABLE 1 lists the electroluminescence characteristics of OLEDs having emitter layers of a benzoxazinone or quinazolinone derivative doped into a CBP host compound according to an aspect of the present invention. The representative stack structure is ITO(1500 Å)/TPD(400 Å)/CBP+x % dopant(400 Å)/BCP(100 Å)/Alq₃(300 Å)/LiF(10 Å)/Cathode. The cathode is either a single layer of aluminum, typically 1000-1500 Å thick, or a layer of aluminum followed with a layer of silver, with the same total thickness. The data are for illustrative purposes only, and are not intended to limit the scope of the invention, which is defined solely by the appended claims. Performance of OLEDs described in terms of brightness, efficiency, driving voltage and current, color coordinates may be changed, i.e. decreased or significantly increased, sometimes by an order of magnitude or more, by varying dopant concentration, layer thickness, layer sequence, and the selection of materials for the different functional layers. All such variations may cumulatively improve energy balance, hole and electron injection, flow of carriers, and exciton-recombination conditions, among other physical conditions, and thus increase the performance of the device.

TABLE 1

| | | | Wavelength | Max. | Current @ | Voltage @ | | |
|---|---|---|---|---|---|---|---|---|
| | | Doping | @ Emission | Luminance | Max. Lum. | Max. Lum. | CIEx @ | CIEy @ |
| Chemical Name | | (wt. %) | Peak (nm) | (cd/m²) | (mA/cm²) | (V) | 20 mA/cm² | 20 mA/cm² |
| 2,2′-(1,3-phenylene)bis-4H-3,1- | 1,3 PBBO | 1.10 | 432 | 1256 | 483 | 15.2 | 0.181 | 0.174 |
| benzoxazinon-4-one | | | | | | | | |
| 2,2′-(4,4′-biphenylene)bis-4H-3,1- | 4,4 BPBBO | 2.30 | 448 | 1167 | 350 | 15.2 | 0.185 | 0.206 |
| benzoxazinon-4-one | | | | | | | | |
| 2,2′,2″-(1,3,5-phenylene)tris-4H-3,1- | 1,3,5 PTBO | 1.18 | 464 | 659 | 286 | 14.8 | 0.188 | 0.244 |
| benzoxazinon-4-one | | | | | | | | |
| 2,2′-(1,4-naphthalyene)bis-4H-3,1- | 2,6 NBBO | 1.30 | 468 | 1527 | 395 | 16.4 | 0.196 | 0.267 |
| benzoxazinon-4-one | | | | | | | | |
| 3H,3′H-[2,2′]-1,4-phenylene-bis- | 1,4 PBQO | 1.40 | 492 | 872 | 373 | 16.0 | 0.232 | 0.346 |
| quinazolin-4-one | | | | | | | | |
| 2,2′-(1,4-phenylene)bis-4H-3,1- | 1,4 PPO | 2.00 | 496 | 60 | 60 | 16.2 | 0.241 | 0.394 |
| benzoxazinon-4-one | | | | | | | | |
| 2,2′-(1,4-naphthaylene)bis-4H-3,1- | 1,4 NBBO | 1.30 | 520 | 1357 | 338 | 16 | 0.277 | 0.430 |
| benzoxazinon-4-one | | | | | | | | |
| [2,2′]bi-[benz[d][1,3]oxazinyl]- | 2,2 BBO | 1.40 | 528 | 1427 | 336 | 13.4 | 0.329 | 0.489 |
| 4,4′-dione] | | | | | | | | |
| 2,2′-(1,4-phenylene-2,3,5,6-tetra- | 1,4 PTFBBO | 1.96 | 532 | 1742 | 339 | 14.8 | 0.341 | 0.500 |
| fluoro)bis-4H-3,1-benzoxazinon- | | | | | | | | |
| 4-one | | | | | | | | |

The following examples are for illustrative purposes only, and are not intended to limit the scope of the invention, which is defined solely by the appended claims.

### EXAMPLES

### Example 1

In this example, a glass substrate with indium-tin-oxide (ITO) film having a thickness of 1500 Å was placed into a rotational substrate holder inside a vacuum deposition chamber with the ITO layer facing a plurality of deposition boats containing materials to be deposited. A 400 Å film of TPD was deposited through a first shadow mask having a square window overlapping the OLED pixel areas onto the ITO anode layer at a rate of 1-2 Å/s to form a hole transport layer (HTL). Thereafter, a 300 Å emitting layer (EML) was deposited via co-deposition of a dopant 2,2′-(1,4-phenylene)bis-4H-3,1-benzoxazin-4-one (1,3 PBBO) and a host compound, carbazole biphenyl (CBP), by means of thermal vapor deposition, through the same shadow mask. To achieve a 2.3% doping level, the deposition rate was maintained at about 0.05 Å/s for the dopant and at about 2.4 Å/s for the host during the course of deposition. For the former, control over such a low rate is usually difficult. Therefore, to improve control, the sensitivity of a thickness monitor quartz balance was set to a factor of ten times higher than usual, and thus the real time monitoring of the thickness was 0.5 Å/s, while the real rate was still 0.05 Å/s. Maintaining the deposition rate at the aforementioned ratio provided a weight ratio of 2.3:97.7 for dopant and host, respectively. A 100 Å hole blocking layer (HBL) of BCP was further deposited through the same shadow mask, followed by deposition of a 400 Å electron transport layer (ETL) of Alq₃. Thereafter, the first shadow mask having a rectangular window was replaced with a second shadow mask having several smaller windows comprising OLED pixels. A 10 Å film of LiF was thereafter deposited onto the electron transport layer as an HIL through the second shadow mask. A 1325 Å layer of aluminum was subsequently deposited as a cathode layer onto the HIL through the second shadow mask.

The OLED thus fabricated having the following stack structure: ITO(1500 Å)/TPD(400 Å)/CBP+2.3% 1,3PBBO(300 Å)/BCP(100 Å)/Alq₃(300 Å)/LiF(10 Å)/Al(1325 Å) was transferred into inert-atmosphere dry box, where it was encapsulated for further characterization with a blank piece of glass coupled to the substrate by an adhesive means. Electroluminescence data were collected by means of Keithley 238 High Current Source Measure Unit and Photoresearch Spectroscan PR650 Spectrophotometer. A special software program written in LabView computer language was used to accelerate data collection and analysis. The data obtained for the device of Example 1 are presented in FIGS. 2a, 2b, and 2c.

### Example 2

A series of OLEDs were fabricated in this example, where similarly to Example 1, glass substrates with indium-tin-oxide (ITO) film having a thickness of 1500 Å were placed into a rotational substrate holder inside a vacuum deposition chamber with the ITO layer facing a plurality of deposition boats containing materials to be deposited. A 400 Å film of TPD was deposited through a first shadow mask having a rectangular window overlapping the OLED pixel areas onto the ITO anode layer at a rate of 1-2 Å/s to form a hole transport layer (HTL). Thereafter, about a 300 to 400 Å thick emitting layer (EML) was deposited via co-deposition of a dopant 2,2′-(1,3-phenylene)bis-4H-3,1-benzoxazin-4-one (1,3 PBBO) and a host compound, carbazole biphenyl (CBP), by means of thermal vapor deposition, through the same shadow mask. During the course of deposition, the evaporation rates are maintained in such way that various doping concentrations ranging from 0 wt. % to 2.3 wt. % were achieved. A 100 Å hole blocking layer (HBL) of BCP was further deposited through the same shadow mask, followed by deposition of a 400 Å electron transport layer (ETL) of Alq₃. Thereafter, the first shadow mask having a rectangular window was replaced with a second shadow mask having several smaller windows comprising OLED pixels. A 10 Å film of LiF was thereafter deposited onto the electron transport layer as an HIL through the second shadow mask. A thick layer of aluminum, typically 1200-200 Å was subsequently deposited as a cathode layer onto the HIL through the second shadow mask.

The series of OLEDs thus fabricated having the following stack structure: ITO(1500 Å)/TPD(400 Å)/CBP+x %1,3PBBO(300-400 Å)/BCP(100 Å)/Alq₃(300-350 Å)/LiF(10 Å)/Al(1200-2000 Å), where x is the emitter dopant concentration, were transferred into inert-atmosphere dry box, where they were encapsulated for further characterization with a blank piece of glass coupled to the substrate by an adhesive means. Electroluminescence data were collected by same means as in Example 1. The data obtained for the devices of Example 2 are presented in FIGS. 3 and 4.

### Example 3

This example is similar to most of the fabrication steps to that of Example 1, except a 400 Å EML was deposited via co-deposition of a dopant, 2,2′-(1,4-phenylene)bis-4H-3,1-benzoxazin-4-one (1,4 PPO), and a CBP host. The deposition rate was maintained at about 0.04 Å/s. for the dopant and at about 1.0 Å/s for the host during the course of deposition to achieve a 3.9 wt. % doping level. As in Example 1, to improve the control, the sensitivity of a thickness monitor was set to a factor of ten times higher than usual, and thus the real-time monitoring thickness was 0.4 Å/s, while the real deposition rate was still 0.04 Å/s. Maintaining the deposition rate at the aforementioned ratio provides a weight ratio of 3.9:96.1 for dopant and host, respectively. A 100 Å hole blocking layer (HBL) of BCP was further deposited through the same shadow mask, followed by deposition of a 350 Å electron transport layer (ETL) of Alq₃. Thereafter, the first shadow mask having a rectangular window was replaced with a second shadow mask having several smaller windows comprising OLED pixels. A 10 Å film of LiF was thereafter deposited onto the electron transport layer as an HIL through the second shadow mask. A 1915 Å layer of aluminum was subsequently deposited as a cathode layer onto the HIL through the second shadow mask.

The OLED thus fabricated having the following stack structure: ITO(1500 Å)/TPD(400 Å)/CBP+3.9% 1,4PPO(400 Å)/BCP(80 Å)/Alq₃(350 Å)/LiF(10 Å)/Al(1915 Å) was transferred into inert-atmosphere dry box, where it was encapsulated for further characterization with a blank piece of glass coupled to the substrate by an adhesive means. Electroluminescence data obtained for the device of Example 3 are presented in FIGS. 5a and 5b.

### Example 4

A substrate coated with 1500 Å ITO was placed into a rotational substrate holder inside a vacuum deposition chamber with the ITO film layer facing deposition boats containing materials to be deposited onto the ITO layer. After a 400 Å film of TPD was deposited upon the ITO layer, a 400 Å emitting layer (EL) was formed via co-deposition of 2.2′-(1,4-naphthylene)bis-4H-3,1-benzoxazin-4-one (2.6 NBBO) as dopant material, and CBP as host material. The rate of deposition was maintained at about 0.04 Å/s for the dopant and about 4 Å/s for the host during the course of deposition, which provided a weight ratio of 1.3:98.7 for the dopant and host, respectively. A very low deposition rate for the dopant was maintained via same experimental procedure described in Example 1. Next, a 100 Å film of BCP was deposited onto the host/dopant layer as HBL. A 350 Å film of Alq₃ was subsequently deposited as an ETL. Lithium fluoride, aluminum and silver were subsequently deposited with thicknesses of 10 Å, 100 Å, and 500 Å, respectively. The fabricated OLED had the following stack structure: ITO(1500 Å)/TPD(400 Å)/CBP+1.3%2,6NBBO(400 Å)/BCP(100 Å)/Alq₃(350 Å)/LiF(10 Å)/Al(100 Å)/Ag(500 Å). The device was encapsulated inside an inert-atmosphere dry box, and characterized by means described in Example 1. The data obtained are presented in FIGS. 6a, 6b, and 6c.

### Example 5

The fabrication of the device of this example involved similar steps as in Example 1. After a 415 Å film of TPD was deposited upon the ITO layer having a thickness of 1500 Å, a 400 Å emitting layer (EL) was formed via co-deposition of 2,2′-(4,4′-biphenylene)bis-4H-3,1-benzoxazin-4-one (4,4 BPBBO) as dopant material, and CBP as host material. The rate of deposition was maintained at about 0.4 Å/s for the dopant and about 2 Å/s for the host during the course of deposition, which provided a weight ratio of 2.3:97.7 for the dopant and host, respectively. Next, a 100 Å film of BCP was deposited onto the host/dopant layer as HBL. A 350 Å film of Alq₃ was subsequently deposited as an ETL. Lithium fluoride, aluminum and silver were subsequently deposited with thicknesses of 10 Å, 100 Å, and 500 Å, respectively. The fabricated OLED has the following stack structure: ITO(1500 Å)/TPD(415 Å)/CBP+2.3% 4,4BPBBO(400 Å)/BCP(100 Å)/Alq₃(350 Å)/LiF(I OA)/Al(100 Å)/Ag(500 Å). The OLED was encapsulated in an inert atmosphere glove-box and characterized to obtain data shown in FIGS. 7a, 7b, and 7c.

### Example 6

In this example, an OLED was fabricated implementing simultaneously pulsed lased deposition and thermal evaporation. The emitter material, 2,2′-(1,4-phenylene)bis-4H-3,1-benzoxazinon-4-one (1,4PPO), was co-deposited by means of pulsed laser deposition and the host material, carbazole biphenyl (CBP) was co-deposited by means of thermal vapor evaporation, through the same shadow mask. Other layers constituting an OLED were subsequently deposited by thermal vacuum evaporation techniques. The OLED thus fabricated had the following stack structure: ITO/TPD(500 Å)/CBP+4%1,4PPO(500 Å)/Alq₃(350 Å)/LiF(10 Å)/Al(1160 Å).

Although the invention has been described with regard to the preferred embodiments, the details of the description are not to be construed as a limitation thereof. Various embodiments, changes, modifications, and equivalent substitutions may be made without departing from the spirit and scope thereof, which is defined solely by the appended claims.

What is claimed is:

1. An organic light emitting device, comprising an emitting layer, said emitting layer comprising a compound according to Formula I: 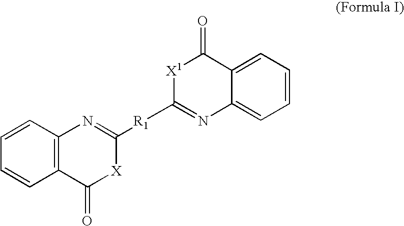
R₁ represents a bond; or from 1 to 3 ring structures, each independently selected from the group consisting of: monocyclic aryls containing 3 to 10 carbon atoms, multicyclic aryls containing 7 to 14 carbon atoms, each monocyclic aryl and multicyclic aryl optionally substituted with one or more hydroxy, halo, acetoxy, or 4H-3,1-benzoxazin-4-one groups; 4 to 10 ring member monocyclic heteroaryls, and 6 to 14 ring member multicyclic heteroaryls, each monocyclic heteroaryl and multicyclic heteroaryl optionally substituted with one or more hydroxy, halo or acetoxy groups.

2. The compound according to claim 1, wherein said monocyclic aryl is a phenyl ring.

3. The compound according to claim 2, wherein said phenyl ring is substituted with fluorine.

4. The compound according to claim 2, wherein said phenyl ring is substituted with hydroxy.

5. The compound according to claim 2, wherein said phenyl ring is substituted with acetoxy.

6. The compound according to claim 2, wherein said phenyl ring is substituted with 4H-3,1-benzoxazin-4-one.

7. The compound according to claim 1, wherein said multicyclic aryl is selected from the group consisting of naphthyl, phenanthryl, and anthracenyl.

8. The compound according to claim 1, wherein said monoheterocyclic aryl is selected from the group consisting of pyridine and pyrimidine.

9. The compound according to claim 1, wherein X is NH and X′ is NH.

10. The compound according to claim 1, wherein X is O and X′ is O.

11. The compound according to claim 1, wherein said halo group is selected from the group consisting of fluorine, chlorine, iodine, and bromine.

12. A compound according to claim 1, wherein the compound is selected from the group consisting of: 2,2′-(1,4-phenylene)bis-4H-3,1-benzoxazin-4-one; 2,2′-(1,4-naphthylene)bis-4H-3,1-benzoxazin-4-one; [2,2′]bi-[benz[d] [1,3]oxazinyl]-4,4′-dione; 2,2′,2″-(1,3,5-phenylene)tris-4H-3,1-benzoxazin-4-one; 2,2′-(1,5-pyridyl)bis-4H-3,1-benzoxazin-4-one; 2,2′-(1,3-phenylene)bis-4H-3,1-benzoxazin-4-one; 2,2′-(1,4-naphthylene)bis-4H-3,1-benzoxazin-4-one; 2,2′-(1,4-phenylene)-2,3,5,6-tetrafluoro)bis-4H-3,1-benzoxazin-4-one; 3H, 3′H-[2,2′]-1,4-phenylene-bis-quinazolin-4-one; 2,2′-(1,4-pyridyl)bis-4H-3,1-benzoxazin-4-one; 2,2′-(1,4-phenylene-2,5-diacetoxy)bis-4H-3,1-benzoxazin-4-one; 2,2′-(1,4-phenylene-2,5-dihydroxy)bis-4H-3,1-benzoxazin-4-one; 3H, 3′H-[2,2′]-biquinazolinyl-4,4′-dione; and 2,2″-(4,4″-biphenylene)bis-4H-3,1-benzoxazinone.

13. A method for emitting electromagnetic radiation with at least one wavelength between about 400 and about 490 nm, comprising the steps of:
providing an organic light emitting device comprising an emitting layer interposed between a hole transport layer and an electron transport layer, said hole transport layer interposed between an anode layer and said emitting layer, and said electron transport layer interposed between a cathode layer and said emitting layer, wherein at least one of said anode layer and said cathode layer is transparent, said emitting layer comprising a compound according to Formula I: 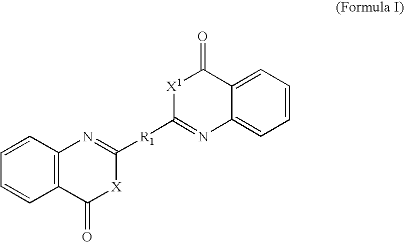
R₁ represents a bond; or from 1 to 3 ring structures, each independently selected from the group consisting of: monocyclic aryls containing 3 to 10 carbon atoms, multicyclic aryls containing 7 to 14 carbon atoms, each monocyclic aryl and multicyclic aryl optionally substituted with one or more hydroxy, halo, acetoxy, or 4H-3,1-benzoxazin-4-one groups; 4 to 10 ring member monocyclic heteroaryls, and 6 to 14 ring member multicyclic heteroaryls, each monocyclic heteroaryl and multicyclic heteroaryl optionally substituted with one or more hydroxy, halo or acetoxy groups; and,
applying an electrical potential to said device between said cathode and anode so that said compound of said emitting layer emits said electromagnetic radiation.

14. An organic light emitting device containing an emitting layer interposed between two electrodes, one of said electrodes being transparent, said device comprising:
an anode electrode layer;
a hole transport layer;
an electron transport layer;
a cathode electrode layer;
an emitting layer interposed between said hole transport layer and said electron transport layer, said hole transport layer interposed between said anode layer and said emitting layer, and said electron transport layer interposed between said cathode layer and said emitting layer, wherein at least one of said anode layer and said cathode layer is transparent, said emitting layer comprising a compound according to Formula I: 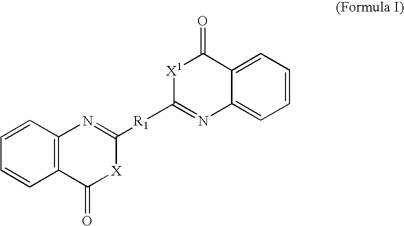
R₁ represents a bond; or from 1 to 3 ring structures, each independently selected from the group consisting of: monocyclic aryls containing 3 to 10 carbon atoms, multicyclic aryls containing 7 to 14 carbon atoms, each monocyclic aryl and multicyclic aryl optionally substituted with one or more hydroxy, halo, acetoxy, or 4H-3,1-benzoxazin-4-one groups; 4 to 10 ring member monocyclic heteroaryls, and 6 to 14 ring member multicyclic heteroaryls, each monocyclic heteroaryl and multicyclic heteroaryl optionally substituted with one or more hydroxy, halo or acetoxy groups;

15. The device according to claim 14, wherein the compound according to Formula I is selected from the group consisting of: 2,2′-(1,4-phenylene)bis-4H-3,1-benzoxazin-4-one; 2,2′-(1,4-naphthylene)bis-4H-3,1-benzoxazin-4-one; [2,2′]bi-[benz[d][1,3]oxa-zinyl]-4,4′-dione; 2,2′,2″-(1,3,5-phenylene)tris-4H-3,1-benzoxazin-4-one; 2,2′-(1,5-pyridyl)bis-4H-3,1-benzoxazin-4-one; 2,2′-(1,3-phenylene)bis-4H-3,1-benzoxazin-4-one; 2,2′-(1,4-naphthylene)bis-4H-3,1-benzoxazin-4-one; 2,2′-(1,4-phenylene)-2,3,5,6-tetrafluoro)bis-4H-3,1-benzoxazin-4-one; 3H, 3′H-[2,2′]-1,4-phenylene-bis-quinazolin-4-one; 2,2′-(1,4-pyridyl)bis-4H-3,1-benzoxazin-4-one; 2,2′-(1,4-phenylene-2,5-diacetoxy)bis-4H-3,1-benzoxazin-4-one; 2,2′-(1,4-phenylene-2,5-dihydroxy)bis-4H-3,1-benzoxazin-4-one; 3H, 3′H-[2,2′]-biquinazolinyl-4,4′-dione; and 2,2″-(4,4″-biphenylene)bis-4H-3,1-benzoxazinone.

16. The device according to claim 14, wherein said emitting layer has an intensity peak wavelength of about 400 to about 490 nm.

17. The device according to claim 14, wherein the emitting layer further comprises 4,4″-N,N′-dicarbazole-biphenyl.

18. The device according to claim 14, wherein the emitting layer consists of 2,2′-(1,4-phenylene)bis-4H-3,1-benzoxazin-4-one and 4,4″-N,N′-dicarbazole-biphenyl in a ratio of 4:9.6.

19. The device according to claim 14, incorporated into a large display for displaying image information.

20. A method for manufacturing the device according to claim 14, comprising:
providing a substrate layer;
disposing a first electrode layer directly on said substrate layer;
disposing a hole transport layer directly on said first electrode layer on the side opposite said substrate layer;
disposing an emitting layer directly on said hole transport layer on the side opposite said first electrode layer, said emitting layer comprising a compound according to Formula I;
disposing an electron transport layer directly on said emitting layer on the side opposite said hole transport layer; and,
disposing a second electrode layer directly on said electron transport layer on the side opposite said electron transport layer, one of said first electrode layer and said second electrode layer being transparent.

21. An emitter composition for organic light emitting devices, comprising a host compound and an emitting compound according to Formula I: 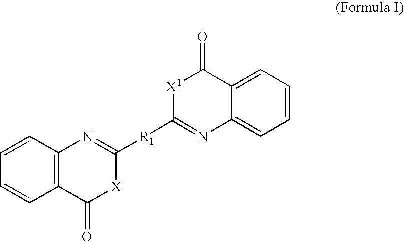
R₁ represents a bond; or from 1 to 3 ring structures, each independently selected from the group consisting of: monocyclic aryls containing 3 to 10 carbon atoms, multicyclic aryls containing 7 to 14 carbon atoms, each monocyclic aryl and multicyclic aryl optionally substituted with one or more hydroxy, halo, acetoxy, or 4H-3,1-benzoxazin-4-one groups; 4 to 10 ring member monocyclic heteroaryls, and 6 to 14 ring member multicyclic heteroaryls, each monocyclic heteroaryl and multicyclic heteroaryl optionally substituted with one or more hydroxy, halo or acetoxy groups.

22. The composition according to claim 21, wherein said host comprises 4,4″-N,N′-dicarbazole-biphenyl.
